SEQUENCE LISTING (18 sequences)

SEQ ID NO: 1 - DNA, 1320 bp, Human rhinovirus
atgggtcatcaccatcatcatcacgggtccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaacgttcttgtacgacggtattgaaattcaagctgatcagacccctgaagatttggacatggaggataacgatattattgaggctcacagagaacagattggaggtggcgcccaggtgagcacccagaagagcggcagccacgagaaccagaacatcctgaccaacggcagcaaccagaccttcaccgtgatcaactactacaaggacgccgccagcaccagcagcgccggccagagcctgagcatggaccccagcaagttcaccgagcccgtgaaggacctgatgctgaagggcgcccccgccctgaacagccccaacgtggaggcctgcggctacagcgaccgggtgcagcagatcaccctgggcaacagcaccatcaccacccaggaggccgccaacgccgtggtgtgctacgccgagtggcccgagtacctgcccgacgtggacgccagcgacgtgaacaagaccagcaagcccgacaccagcgtgtgccggttctacaccctggacagcaagacctggaccaccggcagcaagggctggtgctggaagctgcccgacgccctgaaggacatgggcgtgttcggccagaacatgttcttccacagcctgggccggagcggctacaccgtgcacgtgcagtgcaacgccaccaagttccacagcggctgcctgctggtggtggtgatccccgagcaccagctggccagccacgagggcggcaacgtgagcgtgaagtacaccttcacccaccccggcgagcggggcatcgacctgagcagcgccaacgaggtgggcggccccgtgaaggacgtgatctacaacatgaacggcaccctgctgggcaacctgctgatcttcccccaccagttcatcaacctgcggaccaacaacaccgccaccatcgtgatcccctacatcaacagcgtgcccatcgacagcatgacccggcacaacaacgtgagcctgatggtgatccccatcgcccccctgaccgtgcccaccggcgccacccccagcctgcccatcaccgtgaccatcgcccccatgtgcaccgagttcagcggcatccggagcaagagcatcgtgccccagtgataa

SEQ ID NO: 2 - protein, 438 aa, Human rhinovirus
MGHHHHHHGSLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGGAQVSTQKSGSHENQNILTNGSNQTFTVINYYKDAASTSSAGQSLSMDPSKFTEPVKDLMLKGAPALNSPNVEACGYSDRVQQITLGNSTITTQEAANAVVCYAEWPEYLPDVDASDVNKTSKPDTSVCRFYTLDSKTWTTGSKGWCWKLPDALKDMGVFGQNMFFHSLGRSGYTVHVQCNATKFHSGCLLVVVIPEHQLASHEGGNVSVKYTFTHPGERGIDLSSANEVGGPVKDVIYNMNGTLLGNLLIFPHQFINLRTNNTATIVIPYINSVPIDSMTRHNNVSLMVIPIAPLTVPTGATPSLPITVTIAPMCTEFSGIRSKSIVPQ

SEQ ID NO: 3 - DNA, 1038 bp, Human rhinovirus
atgggtcatcaccatcatcatcacgggtccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaacgttcttgtacgacggtattgaaattcaagctgatcagacccctgaagatttggacatggaggataacgatattattgaggctcacagagaacagattggaggtggcctgcccaccaccaccctgcccggcagcggccagttcctgaccaccgacgaccggcagagccccagcgccctgcccaactacgagcccaccccccggatacacatccccggcaaggtgcacaacctgctggagatcatccaggtggacaccctgatccccatgaacaacacccacaccaaggacgaggtgaacagctacctgatccccctgaacgccaaccggcagaacgagcaggtgttcggcaccaacctgttcatcggcgacggcgtgttcaagaccaccctgctgggcgagatcgtgcagtactacacccactggagcggcagcctgcggttcagcctgatgtacaccggccccgccctgagcagcgccaagctgatcctggcctacaccccccccggcgcccggggcccccaggaccggcgggaggccatgctgggcacccacgtggtgtgggacatcggcctgcagagcaccatcgtgatgaccatcccctggaccagcggcgtgcagttccggtacaccgaccccgacacctacaccagcgccggcttcctgagctgctggtatcagaccagcctgatcctgccccccgagaccaccggccaggtgtacctgctgagcttcatcagcgcctgccccgacttcaagctgcggctgatgaaggacacccagaccatcagccagaccgtggccctgaccgagtgataa

SEQ ID NO: 4 - protein, 344 aa, Human rhinovirus
MGHHHHHHGSLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGGLPTTTLPGSGQFLTTDDRQSPSALPNYEPTPRIHIPGKVHNLLEIIQVDTLIPMNNTHTKDEVNSYLIPLNANRQNEQVFGTNLFIGDGVFKTTLLGEIVQYYTHWSGSLRFSLMYTGPALSSAKLILAYTPPGARGPQDRREAMLGTHVVWDIGLQSTIVMTIPWTSGVQFRYTDPDTYTSAGFLSCWYQTSLILPPETTGQVYLLSFISACPDFKLRLMKDTQTISQTVALTE

SEQ ID NO: 5 - DNA, 1197 bp, Human rhinovirus
atgggtcatcaccatcatcatcacgggtccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaacgttcttgtacgacggtattgaaattcaagctgatcagacccctgaagatttggacatggaggataacgatattattgaggctcacagagaacagattggaggtggcctgggcgacgagctggaggaggtgatcgtggagaagaccaagcagaccgtggccagcatcagcagcggccccaagcacacccagaaggtgcccatcctgaccgccaacgagaccggcgccaccatgcccgtgctgcccagcgacagcatcgagacccggaccacctacatgcacttcaacggcagcgagaccgacgtggagtgcttcctgggccgggccgcctgcgtgcacgtgaccgagatccagaacaaggacgccaccggcatcgacaaccaccgggaggccaagctgttcaacgactggaagatcaacctgagcagcctggtgcagctgcggaagaagctggagctgttcacctacgtgcggttcgacagcgagtacaccatcctggccaccgccagccagcccgacagcgccaactacagcagcaacctggtggtgcaggccatgtacgtgccccccggcgcccccaaccccaaggagtgggacgactacacctggcagagcgccagcaaccccagcgtgttcttcaaggtgggcgacaccagccggttcagcgtgccctacgtgggcctggccagcgcctacaactgcttctacgacggctacagccacgacgacgccgagacccagtacggcatcaccgtgctgaaccacatgggcagcatggccttccggatcgtgaacgagcacgacgagcacaagaccctggtgaagatccgggtgtaccaccgggccaagcacgtggaggcctggattccccgggccccccgggccctgccctacaccagcatcggccggaccaactaccccaagaacaccgagcccgtgatcaagaagcggaagggcgacatcaagagctactgataa

SEQ ID NO: 6 - protein, 397 aa, Human rhinovirus
MGHHHHHHGSLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGGLGDELEEVIVEKTKQTVASISSGPKHTQKVPILTANETGATMPVLPSDSIETRTTYMHFNGSETDVECFLGRAACVHVTEIQNKDATGIDNHREAKLFNDWKINLSSLVQLRKKLELFTYVRFDSEYTILATASQPDSANYSSNLVVQAMYVPPGAPNPKEWDDYTWQSASNPSVFFKVGDTSRFSVPYVGLASAYNCFYDGYSHDDAETQYGITVLNHMGSMAFRIVNEHDEHKTLVKIRVYHRAKHVEAWIPRAPRALPYTSIGRTNYPKNTEPVIKKRKGDIKSY

SEQ ID NO: 7 - protein, 14 aa, Human rhinovirus
PILTANETGATMPV

SEQ ID NO: 8 - protein, 14 aa, Human rhinovirus
PALDAAETGHTSSV

SEQ ID NO: 9 - protein, 14 aa, Human rhinovirus
PILDAAETGHTSNV

SEQ ID NO: 10 - protein, 14 aa, Human rhinovirus
QALGAVEIGATADV

SEQ ID NO: 11 - protein, 16 aa, Human rhinovirus
GAQVSTQKSGSHENQN

SEQ ID NO: 12 - protein, 16 aa, Human rhinovirus
GAQVSRQNVGTHSTQN

SEQ ID NO: 13 - protein, 16 aa, Human rhinovirus
GAQVSRQSVGSHETMI

SEQ ID NO: 14 - protein, 330 aa, Human rhinovirus
GAQVSTQKSGSHENQNILTNGSNQTFTVINYYKDAASTSSAGQSLSMDPSKFTEPVKDLMLKGAPALNSPNVEACGYSDRVQQITLGNSTITTQEAANAVVCYAEWPEYLPDVDASDVNKTSKPDTSVCRFYTLDSKTWTTGSKGWCWKLPDALKDMGVFGQNMFFHSLGRSGYTVHVQCNATKFHSGCLLVVVIPEHQLASHEGGNVSVKYTFTHPGERGIDLSSANEVGGPVKDVIYNMNGTLLGNLLIFPHQFINLRTNNTATIVIPYINSVPIDSMTRHNNVSLMVIPIAPLTVPTGATPSLPITVTIAPMCTEFSGIRSKSIVPQ

SEQ ID NO: 15 - protein, 289 aa, Human rhinovirus
GLGDELEEVIVEKTKQTVASISSGPKHTQKVPILTANETGATMPVLPSDSIETRTTYMHFNGSETDVECFLGRAACVHVTEIQNKDATGIDNHREAKLFNDWKINLSSLVQLRKKLELFTYVRFDSEYTILATASQPDSANYSSNLVVQAMYVPPGAPNPKEWDDYTWQSASNPSVFFKVGDTSRFSVPYVGLASAYNCFYDGYSHDDAETQYGITVLNHMGSMAFRIVNEHDEHKTLVKIRVYHRAKHVEAWIPRAPRALPYTSIGRTNYPKNTEPVIKKRKGDIKSY

SEQ ID NO: 16 - protein, 262 aa, Human rhinovirus
SPNVEACGYSDRVQQITLGNSTITTQEAANAVVCYAEWPEYLPDVDASDVNKTSKPDTSVCRFYTLDSKTWTTGSKGWCWKLPDALKDMGVFGQNMFFHSLGRSGYTVHVQCNATKFHSGCLLVVVIPEHQLASHEGGNVSVKYTFTHPGERGIDLSSANEVGGPVKDVIYNMNGTLLGNLLIFPHQFINLRTNNTATIVIPYINSVPIDSMTRHNNVSLMVIPIAPLTVPTGATPSLPITVTIAPMCTEFSGIRSKSIVPQ

SEQ ID NO: 17 - protein, 236 aa, Human rhinovirus
GLPTTTLPGSGQFLTTDDRQSPSALPNYEPTPRIHIPGKVHNLLEIIQVDTLIPMNNTHTKDEVNSYLIPLNANRQNEQVFGTNLFIGDGVFKTTLLGEIVQYYTHWSGSLRFSLMYTGPALSSAKLILAYTPPGARGPQDRREAMLGTHVVWDIGLQSTIVMTIPWTSGVQFRYTDPDTYTSAGFLSCWYQTSLILPPETTGQVYLLSFISACPDFKLRLMKDTQTISQTVALTE

SEQ ID NO: 18 - protein, 68 aa, Human rhinovirus
GAQVSTQKSGSHENQNILTNGSNQTFTVINYYKDAASTSSAGQSLSMDPSKFTEPVKDLMLKGAPALN

(19) United States
(12) Patent Application Publication
(10) Pub. No.: US 2017/0290905 A1
(43) Pub. Date: Oct. 12, 2017

(54) VACCINE

(71) Applicant: GLAXOSMITHKLINE BIOLOGICALS S.A., Rixensart (BE)

(72) Inventors: Cindy CASTADO, Rixensart (BE); Steve LABBE, Laval (CA); Patrick RHEAULT, Laval (CA)

(73) Assignee: GLAXOSMITHKLINE BIOLOGICALS S.A., Rixensart (BE)

(21) Appl. No.: 15/511,395

(22) PCT Filed: Sep. 17, 2015
(86) PCT No.: PCT/EP2015/071288

§ 371 Date: Mar. 15, 2017

Related U.S. Application Data
(60) Provisional application No. 62/052,145, filed on Sep. 18, 2014.

(51) Int. Cl. A61K 39/125; C12N 7/00

(52) U.S. Cl. CPC A61K 39/125; C12N 7/00; A61K 2039/5258

(57) ABSTRACT

The present invention relates to Human Rhinovirus (HRV) Virus-Like Particles (VLPs) and methods of making HRV VLPs.

### BACKGROUND

The present disclosure relates to the field of human vaccines. More particularly, the present disclosure relates to pharmaceutical and immunogenic compositions, for the prevention or treatment of human infection or disease, in particular human rhinovirus (HRV) infection or disease.

Rhinoviruses are non-enveloped viruses and are composed of a capsid formed from four viral proteins VP1, VP2, VP3 and VP4. VP1, VP2, and VP3 form the major part of the protein capsid. The much smaller VP4 protein of approximately 70 amino acids in length has a more extended structure, and lies at the interface between the capsid and the RNA genome. The capsid is composed of 60 copies of each of these proteins assembled as an icosahedron.

The rhinovirus genome consists of a linear, single-stranded, positive sense RNA of between 7.2 and 8.5 kb in length. Structural proteins are encoded in the 5′ region of the genome (starting from the 5′ end: VP4, VP2, VP3 and VP1) and nonstructural at the 3′ end, as is the case for all picornaviruses. The RNA is translated into a single polyprotein that is cleaved co-translationally and post-translationally into the four structural proteins and seven non structural proteins. The non structural genes are involved in processing the viral genome, viral replication, and shutting down the host cell protein production.

Currently there are over 100 HRV serotypes. Based on nucleotide identity and susceptibility of antiviral compounds HRVs have been classified into clades A, B, C and possibly D (Rollinger & Schmidtke, 2011; Palmenberg, Rathe & Liggett, 2010), see Table below.

| | Receptor | Serotypes | Serotypes | |
|---|---|---|---|---|
| Clades | type | numbers | examples | Remarks |
| HRV-A | Major | 62 | 16 | |
| HRV-A | Minor | 12 | 1A, 1B, 2, 23, 25, | |
| | | | 29, 30, 31, 44, 47, | |
| | | | 49, 62 | |
| HRV-B | Major | 25 | 3, 14 | |
| HRV-C | ? | 7 | | New, emerging, clade |
| HRV87 | ? | 1 | | Same virus as Human Enterovirus 68, of |
| | | | | species HEV-D (Blomqvist et al., 2002) |
| (HRV-D) | Major | 3 | 8, 45, 95 | Potentially separated clade (distinct from |
| | | | | other clades based on VP3 and non- |
| | | | | structural proteins but not VP1 and VP4) |

In addition host cell receptor specificity has been used to further classify these viruses into major and minor groups. Serotypes that use the intercellular adhesion molecule 1 (ICAM-1) receptor (62 HRV-A serotypes and all the B serotypes) belong to the major receptor group and the remaining 12 HRV-A serotypes use members of the low-density lipoprotein (LDL) receptor family and belong to the minor receptor group. Therefore the terms “HRV-A major”, “HRV-A minor”, and “HRV-B major” are used.

Serotypes are further classified by the antigenic sites they utilise to evade the host's immune system. For the major receptor group four primary neutralising immunogenic (NIm) sites have been mapped to protruding regions on the external capsid proteins VP1, VP2 and VP3. These are known as NIm-IA, NIm-IB, NIm-II and NIm-III. For the minor receptor serotypes there are three distinct antigenic sites A, B and C that are located in the same vicinity as the NIm sites (reviewed in Lewis-Rogers et al 2009 Mol Biol Evol 26:969).

Rhinoviruses are the primary cause of acute upper respiratory tract infections in humans, known as the common cold. They are also the most common viral cause of severe exacerbation of chronic respiratory diseases such as asthma and chronic obstructive pulmonary disease (COPD).

The provision of a vaccine against HRV is a particular challenge due to the large number of serotypes of the virus and the lack of a protective response generated in individuals infected with one serotype against infection with another serotype.

No vaccine has so far been developed. A rhinovirus vaccine, which would need to be able to protect against multiple serotypes, therefore represents a large unmet medical need.

One approach under evaluation is the provision of peptides which are conserved among HRV serotypes. It has been demonstrated that antibodies induced with recombinant HRV-14 or -89 VP1 proteins or a peptide spanning amino acids 147-162 of HRVI4 VP1 exhibit specific and cross-neutralizing activity (McCray & Werner, 1998 Nature 329:736; Edlmayr et al., 2011 Eur. Respir 37:44). Antibodies raised against the N terminal 30 amino acids of VP4 were found to neutralise HRV14, HRV16 and HRV29. In addition, antibodies raised to a consensus sequence of the first 24 residues from rhinovirus VP4 also had some cross-neutralising activity (Katpally et al, 2009 J. Virol 83:7040).

WO 2006/078648 relates to peptide vaccines against HRV derived from the transiently exposed regions of VP4 in particular amino acids 1-31 or 1-24 of VP4; WO 2011/050384 relates to peptides from the N terminus of VP1 including amino acids 1-8; WO 2008/057158 relates to NIm IV of rhinovirus, in particular a peptide comprising amino acids 277-283 or 275-285 from the carboxyl terminal region of VP1, in particular from HRV-14.

Virus-Like Particles (VLPs) are an attractive vehicle for antigen presentation, as they provide antigens in a structural environment similar to the structural environment in the virus, yet are not infectious. However, strategies for producing VLPs or sub-VLPs (protomers, pentamers or else) that have worked for other picornaviruses, such as enterovirus (Chung et al. 2010 Vaccine 28, 6951) and FMDV (Porta et al. 2013 J Virol Methods 187:406) have failed for human rhinoviruses.

### BRIEF SUMMARY

Using a novel method, wherein capsid proteins were expressed as fusion proteins with SUMO sequences, the inventors have now been able to generate rhinoviral VLPs.

Accordingly in a first aspect, the invention relates to a virus-like particle (VLP) of rhinovirus.

In a further aspect, the invention relates to an immunogenic composition comprising a VLP of rhinovirus and a pharmaceutically acceptable diluent, excipient or carrier.

In a further aspect, the invention relates to the use of an immunogenic composition as described herein in the manufacture of a medicament for the prevention or treatment of rhinoviral infection, such as prevention or treatment of common cold.

In a further aspect, the invention relates to a method for inducing an immune response, such as an immune response involving neutralising antibodies and/or a cellular response, against rhinovirus in humans comprising administering to a human an immunogenic composition as described herein.

In a further aspect, the invention relates to a method for inducing an immune response, such as an immune response involving cross-neutralising antibodies and/or a cellular response, against rhinovirus in humans comprising administering to a human an immunogenic composition as described herein.

In a further aspect, the invention relates to a method for preventing rhinovirus infection or disease related to rhinovirus, which method comprises administering to a human an immunogenic composition as described herein:

In a further aspect, the invention relates to the use of an immunogenic composition as described herein, in the manufacture of a medicament for the prevention or treatment of COPD or asthma exacerbations.

In a further aspect, the invention relates to a method for producing rhinoviral VLPs comprising the steps of:
a. producing constructs encoding capsid proteins required for the formation of a VLP, wherein each of the capsid proteins is encoded as a fusion protein with a chaperone protein, such as SUMO,
b. co-expressing said constructs in the same host cell or expressing one or more of said constructs in separate host cells, and
c. lysing said host cells and bringing, in case of separate expression, all capsid proteins required for a VLP together.

### BRIEF DESCRIPTION OF THE DRAWINGS

FIG. 1: Anti-His Western blot showing expression and solubility of individually and co-transfected His-SUMO-VPs.

FIG. 2: TEM after negative staining of VLP-VP-SUMO samples.

FIG. 3: Coomassie-stained LDS-PAGE showing molecular weight shift after SUMO-protease treatment of semi-purified SUMO-VPs fusion proteins.

FIG. 4: Western blot (anti-VP1 antibody) showing molecular weight shift before (A) and after (B) SUMO-protease treatment of His-SUMO-VP1 fusion protein.

### DETAILED DESCRIPTION

### Definitions

The term “virus like particle” (VLP) refers to a viral capsid which resembles the external protein structure of the native virus but is non-infectious because it does not contain viral genetic material. Typically, the size of the VLP is similar to that of the virus and/or its structure is icosahedral, composed of repeated identical protein subunits known as capsomeres.

References to amino acid positions, when used herein, refer to positions in capsid proteins of HRV14 (set forth in SEQ ID NOs:14 to 18), or to structurally equivalent positions in capsid proteins of other HRV serotypes. Such structurally equivalent positions are positions that align with a given position in the most optimal alignment between the other serotype capsid protein sequence and that of the HRV14 sequence. For example, position 33 in a given serotype may be structurally equivalent to position 32 in HRV14 if these positions align in the most optimal alignment of the two protein sequences.

The terms “modify” or “modification” where used herein in connection with a capsid protein indicate a modification of the protein as compared to the sequence of that protein found in nature. A modification is preferably a deletion, substitution or insertion of an amino acid, i.e. deletion, substitution or insertion of one or more amino acid. However, the modification may alternatively be a post-translational modification, such as a modification masking a region of the protein. It will be understood, that in the context of this disclosure, there are numerous naturally occurring serotypes of HRV (and HRV proteins), e.g., obtained from different naturally occurring serotypes of HRV.

“Capsid proteins” of a rhinovirus include VP0, VP1, VP2, VP3 and VP4.

The term “human rhinovirus” abbreviated to HRV refers to any serotype of rhinovirus in the family Picornaviridae which is capable of infecting humans and has been identified or has yet to be identified as a rhinovirus. There are several different ways of grouping HRVs as described herein, and each grouping contains multiple virus “serotypes” or “strains” (e.g., HRV-14, HRV-8, HRV-25, etc.) categorized by genetic similarity.

A “variant” when referring to a nucleic acid or a protein (e.g., a VP1 or VP4 nucleic acid or polypeptide) is a nucleic acid or a protein that differs from a reference nucleic acid or protein. Usually, the difference(s) between the variant and the reference nucleic acid or polypeptide constitute a proportionally small number of differences as compared to the referent. A variant protein can differ from the reference protein to which it is compared by the addition, deletion or substitution of one or more amino acids, or by the substitution of an amino acid analogue.

As used herein, a first amino acid sequence has “x % identity” to a second amino acid sequence means that x % represents the number of amino acids in the first sequence which are identical to their matched amino acids of the second sequence when both sequences are optimally aligned, relative to the total length of the second amino acid sequence. Both sequences are optimally aligned when x is maximum. The alignment and the determination of the percentage of identity may be carried out manually or automatically using BLAST.

The term “epitope” refers to a site on an antigen to which B and/or T cells respond. The “immunodominant epitopes” are those epitopes to which a functionally significant host immune response, e.g., an antibody response or a T-cell response, is primarily made.

An “immunogenic composition” is a composition of matter suitable for administration to a human or animal subject (e.g., in an experimental setting) that is capable of eliciting or inducing a specific immune response, e.g., against a pathogen, such as a rhinovirus. As such, an immunogenic composition includes one or more antigens, for example VLPs as described herein. An immunogenic composition can also include one or more additional components, such as an excipient, carrier, and/or adjuvant. In certain instances, immunogenic compositions are administered to elicit or induce an immune response that protects the subject against symptoms or conditions induced by a pathogen. In the context of this disclosure, the term immunogenic composition will be understood to encompass compositions that are intended for administration to a subject or population of subjects for the purpose of eliciting or inducing a protective or palliative immune response against e.g. HRV (that is, vaccine compositions or vaccines).

An “immune response” is a response of a cell of the immune system, such as a B cell, T cell, or monocyte, to a stimulus. An immune response can be a B cell response, which results in the production of specific antibodies, such as antigen specific neutralizing antibodies. An immune response can also be a T cell response, such as a CD4+ response or a CD8+ response. An immune response is a cross-reactive immune response when it is elicited by an antigen from one serotype and reacts not only with virus from that serotype, but also virus from a different serotype.

“Attenuation” of an epitope, when used herein, refers to modification of an epitope which renders the epitope less immunogenic.

### ASPECTS AND EMBODIMENTS OF THE INVENTION

As described above, in a first aspect, the invention relates to a virus-like particle (VLP) of rhinovirus.

In one embodiment, the VLP is of a human rhinovirus (HRV).

The inventors have been able to produce VLPs comprising VP0, the precursor of VP2 and VP4. Thus, in one embodiment, the VLP comprises capsid proteins VP0, VP1 and VP3. In another embodiment, the VLP comprises capsid proteins VP1, VP2, VP3 and VP4.

The capsid proteins in the VLP may be of one serotype or of different serotypes. Thus, in one embodiment, all capsid proteins originate from the same serotype. In another embodiment, the capsid proteins originate from two or more different serotypes, such as two or more different serotypes selected from the group consisting of: HRV 1B, 2, 3, 8, 10, 14, 26, 29, 31, 39, 47, 61, 62, 63, 66, 77, 97 and 100.

Protein Modifications

The VLP according to the invention may only comprise naturally-occurring capsid proteins, e.g. from a specific serotype mentioned herein. However, in another embodiment, one or more capsid proteins has been modified to make it immunogenically more similar to other serotypes, thus increasing its potential to induce an immune response which will also recognise rhinoviruses of other serotypes.

Thus, in one embodiment, one or more of the capsid proteins comprises at least one modification which increases the ability of the VLP to induce a cross-reactive immune response against multiple serotypes, as compared to a VLP wherein said modification is not present. Such a cross-reactive immune response may involve cross-reactive antibodies and/or cross-reactive cellular responses.

In one embodiment, the modification consists of insertion of a peptide from a rhinoviral capsid protein, wherein said peptide is capable of inducing a cross-reactive immune response against two or more serotypes. Typically, the inserted peptides comprises sequences that are conserved between multiple serotypes. The insertion site for the peptide should be chosen so that they do not prevent the formation of VLPs. Formation of VLPs can be tested as described in the Examples herein.

In a further embodiment hereof, said VLP (i.e. the capsid proteins of the VLP) comprises 2, 3, 4 or more insertions of peptides from rhinoviral capsid proteins, optionally from 2 or more different capsid proteins, e.g. insertion of a peptide from VP1 and insertion of a peptide from VP4. In a further embodiment, one, more or all of said peptides consist of fewer than 50 amino acids of the capsid protein, such as between 10 and 30, e.g. between 8 and 18 amino acids of the capsid protein. In an even further embodiment, one of the peptides is amino acids 32-45 from VP1 or a variant of amino acids 32-45 of VP1 having 1-4 amino acid additions or deletions at either end and/or 1-2 amino acid substitutions or additions or deletions within the peptide sequence. In a specific embodiment, the peptide is selected from:

HRV14 (B):
[SEQ ID NO: 7]
32-PILTANETGATMPV-45
HRV8 (A-M):
[SEQ ID NO: 8]
32-PALDAAETGHTSSV-45
HRV25 (A-m):
[SEQ ID NO: 9]
32-PILDAAETGHTSNV-45
HRV_C_026:
[SEQ ID NO: 10]
32-QALGAVEIGATADV-45

or a variant thereof having 1-4 amino acid additions or deletions at either end and/or 1-2 amino acid substitutions or additions or deletions within the peptide sequence.

In a further embodiment, one of the peptides is amino acids 1-16 from VP4 or a variant of amino acids 1-16 of VP4 having 1-4 amino acid additions or deletions at either end and/or 1-2 amino acid substitutions or additions or deletions within the peptide sequence. In a specific embodiment, the peptide is selected from:

HRV14 (B):
[SEQ ID NO: 11]
1-GAQVSTQKSGSHENQN-16
HRV100 (A-M):
[SEQ ID NO: 12]
1-GAQVSRQNVGTHSTQN-16
HRV_C_026:
[SEQ ID NO: 13]
1-GAQVSRQSVGSHETMI-16

or a variant thereof having 1-4 amino acid additions or deletions at either end and/or 1-2 amino acid substitutions or additions or deletions within the peptide sequence.

In a further embodiment, one of the peptides is amino acids 147-162 of VP1 (e.g. VP1 of HRV14) or a variant of amino acids 147-162 of VP1 having 1-4 amino acid additions or deletions at either end and/or 1-2 amino acid substitutions or additions or deletions within the peptide sequence,

In a further embodiment, one of the peptides is amino acids 1-30 of VP4 (e.g. VP4 of HRV14) or a variant of amino acids 1-30 of VP4 having 1-4 amino acid additions or deletions at either end and/or 1-2 amino acid substitutions or additions or deletions within the peptide sequence.

In a further embodiment, one of the peptides is amino acids 1-24 of VP4 (e.g. VP4 of HRV14) or a variant of amino acids 1-24 of VP4 having 1-4 amino acid additions or deletions at either end and/or 1-2 amino acid substitutions or additions or deletions within the peptide sequence.

In a further embodiment, one of the peptides is amino acids 1-8 of VP1 (e.g. VP1 of HRV14) or a variant of amino acids 1-8 of VP1 having 1-4 amino acid additions or deletions at either end and/or 1-2 amino acid substitutions or additions or deletions within the peptide sequence.

In a further embodiment, one of the peptides is amino acids 277-283 of VP1 (e.g. VP1 of HRV14) or a variant of amino acids 277-283 of VP1 having 1-4 amino acid additions or deletions at either end and/or 1-2 amino acid substitutions or additions or deletions within the peptide sequence.

In a further embodiment, one of the peptides is amino acids 275-285 of VP1 (e.g. VP1 of HRV14) or a variant of amino acids 275-285 of VP1 having 1-4 amino acid additions or deletions at either end and/or 1-2 amino acid substitutions or additions or deletions within the peptide sequence.

In particular embodiments, amino acid substitutions as referred to herein are conservative substitutions.

Several positions on the capsid protein are suitable for insertion of such peptides.

In one embodiment, the peptide has been inserted in VP0, such as in VP0 of HRV14, e.g. in one or more of the regions corresponding to 72-76, 131-175, 133-145, 158-165, 228-238, 231-237 or 255-262 of VP2.

In a further embodiment, the peptide has been inserted in VP1, such as in VP1 of HRV14, e.g. in one or more of the regions 73-102, 85-92, 129-149, 136-145, 160-168, 204-212, 208-215, 230-236 or 264-289 of VP1.

In a further embodiment, the peptide has been inserted in VP2, such as in VP2 of HRV14, e.g. in one or more of the regions 72-76, 131-175, 133-145, 158-165, 228-238, 231-237 or 255-262 of VP2.

In a further embodiment, the peptide has been inserted in VP3 such as in VP3 of HRV14, e.g. in one or more of the regions 54-95, 57-64, 72-77, 193-212, 196-204 or 226-236 of VP3.

In a further embodiment, the peptide has been inserted in VP4 such as in VP4 of HRV14.

In addition to, or as an alternative for, the insertion of a peptide, the VLP may comprise at least one modification which results in attenuation of an immunodominant epitope. Immunodominant epitopes are often hypervariable and thus by attenuating such epitopes, the immune response may become more directed to other, more conserved, epitopes on the capsid.

In one such embodiment, VP0 comprises said modification, e.g. wherein VP0 has been modified so that the NIm-II site epitope has been attenuated and/or wherein one or more residues in one or more of the regions corresponding to 131-175, 228-238 or 255-262 in VP2 has been modified, e.g. wherein a residue corresponding to residue 158, 159, 161 and/or 162 has been modified.

In another such embodiment, VP1 comprises said modification, e.g. wherein VP1 has been modified so that the NIm-IA site epitope has been attenuated and/or VP1 has been modified so that the NIm-IB site epitope has been attenuated and/or wherein one or more residues in one or more of the regions 73-102, 129-149, 204-212 and 264-289 has been modified, for example wherein residue 91 and/or 95 has been modified or wherein residue 83, 85, 138 and/or 139 has been modified.

In another such embodiment, VP2 comprises said modification, e.g. wherein VP2 has been modified so that the NIm-II site epitope has been attenuated and/or wherein one or more residues in one or more of the regions 131-175, 228-238 or 255-262 has been modified, e.g wherein residue 158, 159, 161 and/or 162 has been modified.

In another such embodiment, VP3 comprises said modification, e.g. wherein VP3 has been modified so that the NIm-III site epitope has been attenuated and/or wherein one or more residues in one or more of the regions 54-95, 193-212 or 226-236 has been modified, e.g. wherein residue 72, 75 and/or 78 has been modified.

In another such embodiment, VP4 comprises said modification.

Regardless of whether the above-described modifications involve insertions, deletions or substitutions of amino acids, the capsid protein sequences will remain similar to the naturally-occurring protein sequences from which they are derived. The VLP may contain a mixture of unmodified and modified capsid proteins.

In one embodiment, the VP0 protein is the HRV14 VP0 protein or a variant thereof, wherein the variant has more than 90% sequence identity to HRV14 VP0.

In another embodiment, the VP0 protein is the HRV14 VP0 protein or a variant thereof having 1-25, such as 1-10 amino acid additions or deletions at either end and/or 1-50, such as 1-25, e.g. 1-15 amino acid substitutions or additions or deletions within the sequence.

In another embodiment, the VP1 protein is the HRV14 VP1 protein or a variant thereof, wherein the variant has more than 90% sequence identity to HRV14 VP1.

In another embodiment, the VP1 protein is the HRV14 VP1 protein or a variant thereof having 1-25, such as 1-10 amino acid additions or deletions at either end and/or 1-50, such as 1-25, e.g. 1-15 amino acid substitutions or additions or deletions within the sequence.

In another embodiment, the VP2 protein is the HRV14 VP2 protein or a variant thereof, wherein the variant has more than 90% sequence identity to HRV14 VP2.

In another embodiment, the VP2 protein is the HRV14 VP2 protein or a variant thereof having 1-25, such as 1-10 amino acid additions or deletions at either end and/or 1-50, such as 1-25, e.g. 1-15 amino acid substitutions or additions or deletions within the sequence.

In another embodiment, the VP3 protein is the HRV14 VP3 protein or a variant thereof, wherein the variant has more than 90% sequence identity to HRV14 VP3.

In another embodiment, the VP3 protein is the HRV14 VP3 protein or a variant thereof having 1-25, such as 1-10 amino acid additions or deletions at either end and/or 1-50, such as 1-25, e.g. 1-15 amino acid substitutions or additions or deletions within the sequence.

In another embodiment, the VP4 protein is the HRVI4 VP4 protein or a variant thereof, wherein the variant has more than 90% sequence identity to HRVI4 VP4.

In another embodiment, the VP4 protein is the HRV14 VP4 protein or a variant thereof having 1-25, such as 1-10 amino acid additions or deletions at either end and/or 1-50, such as 1-25, e.g. 1-15 amino acid substitutions or additions or deletions within the sequence.

As described above, in a further aspect, the invention relates to an immunogenic composition comprising a VLP as described herein and a pharmaceutically acceptable diluent, excipient or carrier. Pharmaceutically acceptable carriers and excipients are well known and can be selected by those of skill in the art. For example, the carrier or excipient can favorably include a buffer. Optionally, the carrier or excipient also contains at least one component that stabilizes solubility and/or stability. Examples of solubilizing/stabilizing agents include detergents, for example, laurel sarcosine and/or tween. Alternative solubilizing/stabilizing agents include arginine, and glass forming polyols (such as sucrose, trehalose and the like). Numerous pharmaceutically acceptable carriers and/or pharmaceutically acceptable excipients are known in the art and are described, e.g., in Remington's Pharmaceutical Sciences, by E. W. Martin, Mack Publishing Co., Easton, Pa., 5th Edition (975).

Accordingly, suitable excipients and carriers can be selected by those of skill in the art to produce a formulation suitable for delivery to a subject by a selected route of administration. Suitable excipients include, without limitation: glycerol, Polyethylene glycol (PEG), Sorbitol, Trehalose, N-lauroylsarcosine sodium salt, L-proline, Non detergent sulfobetaine, Guanidine hydrochloride, Urea, Trimethylamine oxide, KCl, Ca²⁺, Mg²⁺, Mn²⁺, Zn²⁺ and other divalent cation related salts, Dithiothreitol, Dithioerytrol, and β-mercaptoethanol. Other excipients can be detergents (including: Tween80, Tween20, Triton X-00, NP-40, Empigen BB, Octylglucoside, Lauroyl maltoside, Zwittergent 3-08, Zwittergent 3-0, Zwittergent 3-2, Zwittergent 3-4, Zwittergent 3-6, CHAPS, Sodium deoxycholate, Sodium dodecyl sulphate, Cetyltrimethylammonium bromide).

In one embodiment, the composition comprises 2, 3, 4 or more different VLPs of the invention as described herein.

One important aspect of a vaccine against rhinovirus is that it will protect against a sufficient number of rhinovirus serotypes to provide effective protection against rhinovirus infection. Thus, a combination of the above-described embodiments may provide an optimal composition. For example, the immunogenic composition of the invention may contain multiple VLPs originating from different serotypes, and/or VLPs comprising capsin proteins originating from different serotypes, and/or modified capsin proteins wherein one or more conserved peptides have been inserted, and/or modified capsin proteins wherein one or more immunodominant epitopes have been attenuated.

In another embodiment, the immunogenic composition further comprises an adjuvant, such as a Th1 adjuvant. Suitable adjuvants include suspensions of minerals (alum, aluminium hydroxide, aluminium phosphate) onto which antigen is adsorbed; emulsions, including water-in-oil, and oil-in-water (and variants thereof, including double emulsions and reversible emulsions), liposaccharides, lipopolysaccharides, immunostimulatory nucleic acids (such as CpG oligonucleotides), liposomes, Toll-like Receptor agonists (particularly, TLR2, TLR4, TLR7/8 and TLR9 agonists), and various combinations of such components.

In a preferred embodiment, the adjuvant is an aluminium salt, such as aluminium hydroxide. In another preferred embodiment, the adjuvant comprises a TLR4 agonist, such as 3-O-deacylated monophosphoryl lipid A (3D-MPL) and/or a saponin, such as QS21 (WO 96/33739), preferably in a liposomal formulation.

In another embodiment, the immunogenic composition comprises further antigens derived from pathogens other than HRV, such as Moraxella catarrhalis (MCat) or nontypeable Haemophilus influenzae (NTHI). Further inclusion of antigens directed at MCat and/or NTHI are particularly contemplated in the prevention or treatment of COPD.

In one aspect, the invention relates to the VLP or immunogenic composition as described herein for use in the prevention or treatment in humans of a rhinoviral infection, such as prevention or treatment of common cold.

In another aspect, the invention relates to the VLP or immunogenic composition as described herein for use in induction of an immune response involving neutralising antibodies and/or a cellular response, against rhinovirus in humans. In one embodiment the neutralising antibodies are cross-neutralising antibodies.

In another aspect, the invention relates to the use of a VLP or an immunogenic composition as described herein, in the manufacture of a medicament for the prevention or treatment in humans of rhinoviral infection, such as prevention or treatment of common cold.

In another aspect, the invention relates to the use of a VLP or an immunogenic composition as described herein, in the manufacture of a medicament inducing an immune response involving neutralising antibodies and/or a cellular response, against rhinovirus in humans. In one embodiment the neutralising antibodies are cross-neutralising antibodies.

In another aspect, the invention relates to a method for inducing an immune response, such as an immune response involving neutralising antibodies and/or a cellular response, against rhinovirus in humans comprising administering to a human a VLP or an immunogenic composition as described herein.

In another aspect, the invention relates to a method for inducing an immune response, such as an immune response involving cross-neutralising antibodies and/or a cellular response, against rhinovirus in humans comprising administering to a human a VLP or an immunogenic composition as described herein.

In another aspect, the invention relates to a method for preventing rhinovirus infection or disease related to rhinovirus, such as common cold, COPD or asthma exacerbations, which method comprises administering to a human a VLP or an immunogenic composition as described herein.

In another aspect, the invention relates to the VLP or the immunogenic composition as described herein for use in the prevention or treatment of COPD or asthma exacerbations in humans.

In another aspect, the invention relates to the use of a VLP or an immunogenic composition as described herein, in the manufacture of a medicament for the prevention or treatment of COPD or asthma exacerbations in humans.

The immunogenic compositions may be for eliciting an immune response against HRV in human infants (e.g., infants between birth and 1 year, such as between 0 and 6 months, at the age of initial dose). Or in another embodiment, immunogenic compositions may be for eliciting an immune response against HRV in elderly humans. Or the immunogenic composition may be for administration to adults or children. It will be appreciated that the choice of adjuvant can be different in these different applications, and the optimal adjuvant and concentration for each situation can be determined empirically by those of skill in the art.

The immunogenic compositions described herein can be administered as vaccines by any of a variety of routes. Intramuscular, sublingual and intradermal deliveries are preferred.

The dosage of the VLPs can vary with the condition, sex, age and weight of the individual and the administration route of the vaccine. The quantity can also be varied with the number of different VLPs.

Typically, the amount of protein in each dose of the immunogenic composition is selected as an amount which induces an immunoprotective response without significant, adverse side effects in the typical subject. Immunoprotective in this context does not necessarily mean completely protective against infection; it means protection against symptoms or disease, especially severe disease associated with the virus. The amount of antigen can vary depending upon which specific immunogen is employed. Generally, it is expected that each human dose will comprise 1-1000 μg of protein. Suitably each vaccine dose comprises 1-100 μg of each VLP, suitably at least 5 μg, or at least 10 μg, for example, between 5-50 μg of each VLP.

The immunogenic compositions described herein suitably generate an immune response in a human or animal subject against at least 2 different rhinoviruses or two different serotypes of a rhinovirus such as two different HRV serotypes, suitably 2 or more, 3 or more, 4 or more, 5 or more, or 10 or more different HRV serotypes. Cross-protection against different HRV serotypes can e.g. be identified using an animal model, for example mouse models (Bartlett et al 2008).

Suitably the immunogenic composition is delivered in a 2 or 3 dose regimen, for example in a 0, 1 or a 0, 2 or a 0, 3 or a 0, 4 or a 0, 5 or a 0, 6 or a 0, 12 month regimen, or 0, 1, 6 or a 0, 2, 6 or a 0, 6, 12 month regimen respectively.

Methods for Producing Rhinoviral VLPs

As described above, in one aspect, there is provided a method for producing rhinoviral VLPs, such as the VLPs described herein above, said method comprising the steps of:
a. producing constructs encoding capsid proteins required for the formation of a VLP, wherein each of the capsid proteins is encoded as a fusion protein with a chaperone protein,
b. co-expressing said constructs in the same host cell or expressing one or more of said constructs in separate host cells, and
c. lysing said host cells and, in case of separate expression, bringing together all capsid proteins required for a VLP.

The term “chaperone protein” or “chaperone sequence” when used herein refers to a protein that assists other proteins to fold properly and stabilizes proteins. Typically, proper folding and stabilisation leads to improved solubility. Examples of such chaperone proteins are SUMO and Hsp90. In one embodiment, said chaperone protein is SUMO or Hsp90, preferably SUMO. In an alternative embodiment, constructs using SUMO as chaperone and constructs using Hsp90 as chaperone are both co-expressed.

In one embodiment, the method further comprises step d. removing said chaperone protein. In a specific embodiment, the chaperone protein is SUMO. Removal of SUMO in step d. can be, but is not necessarily, done using SUMOstar protease.

Methods for production of nucleic acid constructs are well known in the art. In certain embodiments, the recombinant nucleic acids that encode the proteins are codon optimized for expression in a selected host cell. To facilitate replication and expression, the nucleic acid constructs that encode the proteins can be incorporated into a vector, such as a prokaryotic or a eukaryotic expression vector.

Suitable host cells include prokaryotic (i.e., bacterial) host cells, such as E. coli, as well as numerous eukaryotic host cells, including fungal (e.g., yeast) cells, insect cells, plant cells, and mammalian cells (such as CHO and HEK293 cells). Preferred host cells are eukaryotic cells, e.g. mammalian cells, in particular HEK293 cells.

The host cells can be cultured in conventional nutrient media modified as appropriate for activating promoters, selecting transformants, or amplifying the inserted polynucleotide sequences. The culture conditions, such as temperature, pH and the like, are typically those previously used with the host cell selected for expression, and will be apparent to those skilled in the art and in the references cited herein, including, e.g., Freshney (1994) Culture of Animal Cells, a Manual of Basic Technique, third edition, Wiley-Liss, New York and the references cited therein. In addition to Sambrook, Berger and Ausubel, details regarding cell culture can be found in Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg New York) and Atlas and Parks (eds) The Handbook of Microbiological Media (1993) CRC Press, Boca Raton, Fla.

Typically, the method for producing VLPs as described herein above will further comprise one or more purification steps, e.g. a purification step between steps c. and d. and/or a purification step after step d.

Addition of a tag to the fusion protein can facilitate purification. In one embodiment, the fusion protein comprises a histidine tag. Proteins comprising a histidine tag can be purified using immobilized metal affinity chromatography. Thus, in one embodiment, the method comprises a purification step which comprises immobilized metal affinity chromatography.

The term “Hsp90” when used herein refers to heat shock protein 90. Alternatives include other members of the 90-kDa molecular chaperone family, such as described by Csermely P. et al. (“The 90-kDa Molecular Chaperone Family: Structure, Function, and Clinical Applications. A Comprehensive Review” Pharmacol. Ther. Vol. 79, No. 2, pp. 129-168, 1998).

A “SUMO sequence” or “SUMO” when used herein refers to a Small Ubiquitin-related Modifier sequence or protein. SUMO sequences, like other chaperone proteins, enhance the solubility of expressed fusion proteins. Suitable SUMO sequences have been described in Ulrich “SUMO Protocols” Methods in Mol Biol 497. Humana Press 2009 (herein incorporated by reference) and include SUMO-1, SUMO-2, SUMO-3, SUMO-4, Smt3 and Pmt3. A pET SUMO Expression System for bacterial expression is e.g. available from Life Technologies. SUMO fusion technology has been review by Panavas et al. 2009 Methods Mol Biol 497:303.

In a preferred embodiment, the SUMO sequence is Smt3.

In one embodiment, the SUMO sequence is removed using SUMOprotease.

In one embodiment of the method of the invention, one of the constructs encodes VP0 and said construct encodes the sequence set forth in SEQ ID NO:2.

In another embodiment of the method of the invention, the construct encoding VP3 encodes the sequence set forth in SEQ ID NO:4.

In another embodiment of the method of the invention, the construct encoding VP1 encodes the sequence set forth in SEQ ID NO:6.

### EXAMPLES

Materials and Methods

1. Generation of the Expression Plasmids

Codon Optimization

The Rhinovirus genomic sequence HRV14 from NCBI (accession number NC_001490) was used as a reference. The portion of the HRV14 genomic sequence coding for the P1 structural protein precursor was codon optimized to align with Homo sapiens codon usage table in order to facilitate expression in the human derived selected expression host which is the Human Embryonic Kidney 293 (HEK293) cell line. Although nucleotidic sequence was optimized, none of these substitutions resulted in amino acid sequence alteration.

Construct Assembly

The same procedure applies to the three chimeric SUMO-VPs constructs (SUMO-VP0 (SEQ ID NO:1, encoding SEQ ID NO:2), SUMO-VP3 (SEQ ID NO:3, encoding SEQ ID NO:4) and SUMO-VP1 (SEQ ID NO:5, encoding SEQ ID NO:6)) generated for the experiment.

The DNA fragment coding for the chimeric SUMO-VP was generated by the assembly of two smaller DNA fragments. The first DNA fragment consisted of the entire coding sequence for the SUMOstar fusion protein and a portion of the structural HRV VP protein. The second fragment was PCR amplified from a codon optimized HRV14 DNA template. The two fragments were assembled using PCR.

Molecular Cloning

The assembled fragment and eukaryotic expression vector pTT5 (Zhang et al. (2009) Protein Expr Purif 65:77) were used to generate the expression plasmids. Procedures sufficient to guide one of skill in the art can be found in the following references: Sambrook et al., Molecular Cloning: A Laboratory Manual, Cold Spring Harbor Laboratory Press, Cold Spring Harbor, N.Y., 2000; and Ausubel et al. Short Protocols in Molecular Biology, 4th ed., John Wiley & Sons, Inc., 1999.

2. Description of Protein Expression Conditions

Cell Maintenance

The experiment was conducted in a mammalian expression system. The cells were grown in suspension in F17 animal origin-free, chemically defined, protein-free expression medium (Life technologies, CA, USA) supplemented with 4 mM Glutamine and 0.1% Pluronic F-68 at 37° C., 5% CO₂ and 120 rpm. Passages were done by dilution in fresh medium regularly. Cells were maintained within the 0.2-4.0×10⁶ cells/mL range of viable cell density. Cell density and viability were monitored using the Cellometer cell counter (Nexcelom Bioscience).

Transient Transfection

Following is an example of a transient transfection at a 30 mL scale. Volumes were scaled up accordingly for larger production up to 4 liters. The same culture medium (F17 animal origin-free, chemically defined, protein-free expression medium supplemented with 4 mM Glutamine and 0.1% Pluronic F-68) as in the hereabove cell maintenance section was used for the transfection step.

Briefly, the day before the transfection, a 125 mL cell culture shake flask was seeded with 30 mL of HEK293-6E cells at a 5.0×10⁵ cells/mL viable cell density and were allowed to grow overnight at 37° C., 5% CO₂ 110-120 rpm. On the transfection day, cell density was adjusted at 1.0×10⁶ cells/mL. Transfection mixes were prepared as followed; 12.5 μg of transfection grade expression plasmids His-SUMO-VP0, His-SUMO-VP3 and His-SUMO-VP1 (total of 37.5 ug of expression plasmids) were diluted in Opti-Pro™ SFM buffer (Life technologies, CA, USA) to a total volume of 0.6 mL. Similarly, 37.5 μL of FreeStyle Max transfection reagent was also diluted in Opti-Pro™ SFM to a total volume of 0.6 mL. Diluted DNA and transfection reagent were then mixed together and incubated at room temperature for 10 minutes. After the incubation time, the transfection mix was added slowly to the cells and put back in culture at 37° C., 5% CO₂ 110-120 rpm for a 6-day expression period. At that time, cells were harvested by centrifugation at 6000×g for 10 mins at 4° C.

3. Description of Protein Purification

Cell Lysis

Cell pellet corresponding to 2 L culture, was resuspended in 50 ml of 20 mM Bicine buffer (pH 8.3) containing 0.5M NaCl. Cell suspension was disrupted, using a TS Bench Top Constant Cell Disrupter System 1.1 KW (Constant Systems, Ltd), by two passages at 15 000 PSI (pounds per square inch). Soluble (supernatant) and insoluble (pellet) material were separated by centrifugation at 20 000×g for 20 min at 4° C.

IMAC Purification Step

Soluble material containing the three co-expressed chimeric SUMO-VPs was purified under native conditions on immobilized metal affinity chromatography (IMAC) using protein purification system (PROFINIA™ Bio-Rad Laboratories, Inc. or AKTA system, GE Healthcare, Inc.). The protein preparation was loaded at 2 ml/min on 5 ml His Trap FF columns (GE Healthcare, Inc.) pre-equilibrated with 20 mM Bicine buffer (pH 8.3), 0.5M NaCl, (producing a “flow through fraction”). Thereafter, the column was washed at 10 ml/min flow rate, using 5 column volumes (CV) of the same buffer (producing a “wash fraction #1”), followed by 5CV of the buffer supplemented with 10 mM imidazole (producing a “wash fraction #”2). Elution step was performed using two CV of 20 mM Bicine buffer (pH 8.3), 0.5M NaCl, 250 mM imidazole and at a flow rate of 2 ml/min, producing an “elution fraction”.

SEC Purification Step

A fraction of the IMAC “elution fraction” was loaded on a size exclusion chromatography (SEC) column (HiLoad™ Superdex™ 200 pg, GE Healthcare, Inc) preequilibrated in 20 mM Bicine buffer (pH 8.3) containing 0.5M NaCl, with and without 1 mM TCEP, at 2.5 ml/min. Fractions were analyzed and pooled according to sodium dodecyl sulfate polyacrylamide gel electrophoresis (SDS-PAGE) results. If required, proteins were concentrated using Amicon Ultra Centrifugal Filter Unit-10,000 NMWL (EMD Millipore, Inc.).

4. Use of the SUMOstar Protease

Purified chimeric SUMO-VPs proteins were treated with the SUMOstar protease (SUMOstar kit (cat#7110) from Life Sensors) in order to remove the His-SUMO protein fusion parts and liberate the VP proteins. The SUMOstar protease was added to the protein preparation according to the recommended ratio (1 U/100 μg) in presence of SUMOstar protease buffer and 1 to 5 mM DTT and incubated at 30° C., for more than 60 minutes. Proteolysis experiments could be performed in presence of IMAC resin (IMAC sepharose 6 FF, GE Healthcare, Inc).

5. Description of the Characterisation of the Particles

Sample Preparation for SDS-PAGE

For example, 1 ml of culture was centrifuged at 14 000 RPM for 2 min. The cell pellet was resolubilized using 140 or 210 μl of 8M urea buffer, 10 or 15 μl of dithiothreitol (DTT) 1M and 50 or 75 μl of NUPAGE® LDS (Lithium Dodecyl Sulphate) Sample Buffer 4X (Life Technologies). The cells in suspension were lysed by sonication, with 10 to 15 cycles of 2 seconds of sonication at an output watt of 0.08, amplitude of 40-50, followed by a pause step of one second (Vibra Cell, Sonics & Materials, Inc.). The cell lysates were then centrifuged at 14 000 RPM for 2 min to separate the insoluble components. Thereafter, samples were heated at 70° C. for 10 minutes.

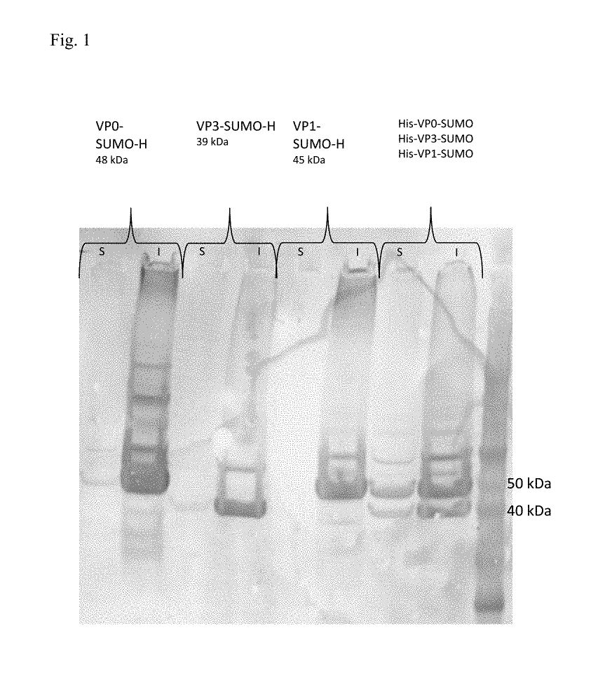

Alternatively, when desired to see the effect of SUMO sequence on the solubility of the co-expressed proteins, for example as in the case for FIG. 1, samples were prepared as follows. Cells contained in 1 ml of culture were isolated by centrifugation at 5000 g for 5 mins. The pelleted cells were resuspended in 200 μl of 20 mM of Bicine and 150 ml NaCl, and, sonicated. Soluble and insoluble protein were separated by centrifugation at 14000 rpm for 10 mins. The pellet obtained was resuspended in 200 μl of 20 mM Bicine, 8M urea, and, sonicated. The soluble part was thus contained in the supernatant.

Purified proteins were prepared for SDS-PAGE analysis by adding 70 ul of sample, 5 ul of DTT 1M and 25 ul of LDS Sample Buffer 4X.

SDS-PAGE Analysis

SDS-PAGE analyses were performed according to manufacturer's recommendations (Life Technologies) using NUPAGE® Bis-Tris 4-12% gels. Preparations of samples, buffers and migration conditions were done under conditions recommended by the suppliers.

DLS

The oligomerization state of VLP-VP-SUMO was evaluated by dynamic light scattering (DLS) which is used to evaluate hydrodynamic radius in solution, to provide information about homogeneity and to detect presence of high molecular weight particles. This is based on the calculation of the diffusion coefficient of the different species that are obtained by measuring the light scattering fluctuation, which depends on protein molecular size and shape, and on the other minor constituents of the sample. Briefly, samples obtained from the SEC purification step were centrifuged at 20 000 g for 2 minutes before being charged in Low volume 384 well black clear flat bottom Corning 3540 plate and subject to DLS analysis using a DynaPro® Plate reader from Wyatt Technology at 25° C. For each sample analyzed by DLS, five consecutive wells were loaded with the same sample and six consecutive measurements were taken on the same well and measured with a light-scattering data collection of 15 s. Analyzing particle size distribution by cumulant or regularization fit and polydispersity index were calculated from the correlation function using Dynamics Software version 7.1.7 (Wyatt Technology).

TEM

Samples obtained from the SEC purification step were prepared for TEM (transmission electron microscopy) negative staining using phosphotungstic acid 3% as contrasting agent and samples were adsorbed on TEM grids (200 mesh) using an Airfuge (an air-driven ultracentrifuge made by Beckman). The material was left to dry completely and examined by transmission electron microscopy (Hitachi H-7100) at 75 kV.

Results

Expression in HEK293

Three expression plasmids (His-SUMO-VP0, His-SUMO-VP3 and His-SUMO-VP1) were used to simultaneously and individually transfect HEK293-6E cells. After the 6 day expression period, the transfected HEK293-6E cells were harvested, resuspended and lysed. Soluble and insoluble fractions were loaded on LDS-PAGE and Western transferred. Western blot analysis using rabbit anti-His polyvalent antibody revealed that individually all 3 His-SUMO-VPs were expressed at a relatively high level but mainly in the insoluble fraction. On the other hand, solubility improved when the 3 His-SUMO-VPs were coexpressed (FIG. 1).

Purification Step 1

Total HEK293 pellet was resuspended in a suitable buffer to keep the proteins in a native condition (no denaturation). The cell suspension was disrupted and phases were separated by centrifugation. The soluble fraction was used to perform a conventional IMAC purification under native conditions.

Purification Step 2

The IMAC purified proteins/particles under native conditions were submitted to an additional purification step. By performing a size exclusion chromatography (SEC) step, we aimed at separating the loaded material based on its size in solution. Samples from different fractions containing His-SUMO-VPs were pooled and characterized for their content of particles.

Dynamic Light Scattering (DLS) and Transmission Electronic Microscopy (TEM)

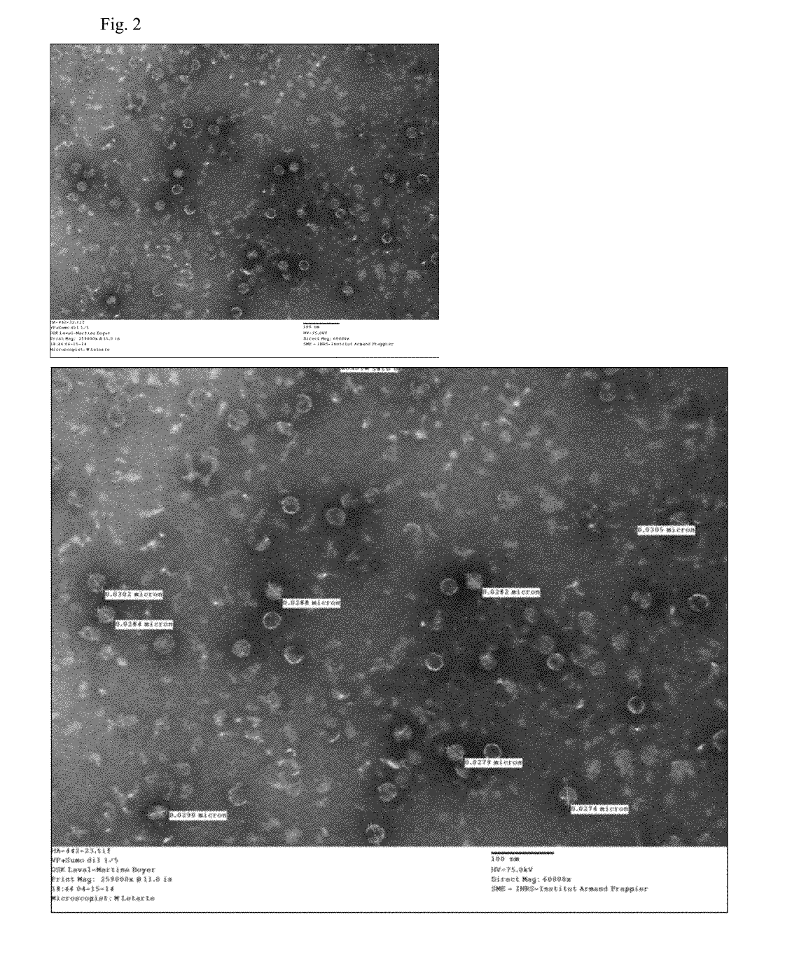

The different pooled fractions were analysed in parallel in DLS (Table 1) and TEM (FIG. 2).

TABLE 1
Diameter results from batch mode Dynamic
light scattering experiment.

| | | Regularization fit | | | |
|---|---|---|---|---|---|
| | Cumulant fit | | | % | |
| | Diameter | Diameter | % | Polydis- | % |
| Sample | (nm) | (nm) | Mass | persity | Intensity |
| His-SUMO-VPs | 37.0 ± 8.6 | 27.6 ± 1.4 | 94.8 | 34.6 | 97.8 |

The measured hydrodynamic diameter of 27.6 nm is coherent with the expected size range of a virus like particle (VLP). Also, the high mass and intensity percentage of the target population and the low hydrodynamic diameter of the overall sample (cumulant fit) suggest a good sample homogeneity.

Both methods gave us good indications that structures were obtained in the range of 30 nm in diameter (Table 2).

TABLE 2
Summary of diameters and widths of distribution
and comparison between TEM and DLS

| | DLS hydrodynamic | TEM external |
|---|---|---|
| | diameter | diameter |
| Sample | Mean (nm) | Mean (nm) |
| His-SUMO-VPs | 27.6 ± 1.4 | 30 ± 3 |

SUMOprotease Processing

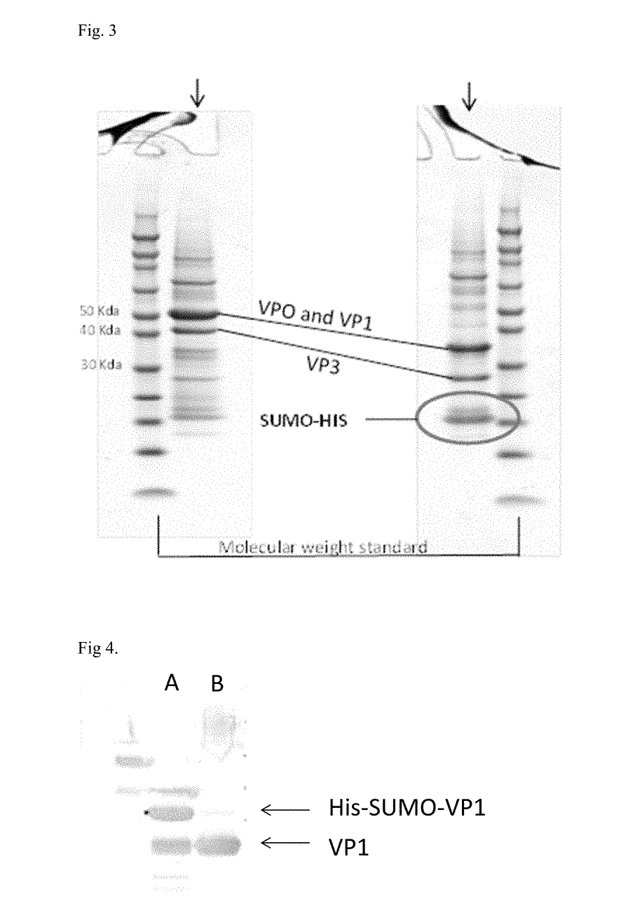

The data described above were generated using the His-SUMO-VPs purified proteins. In vitro digestion of the His-SUMO-VPs proteins using SUMOstar protease was performed to liberate the His-SUMO from the VPs portion. As a result, a clear indication that a good proportion of the His-SUMO-VPs were properly cleaved (into His-SUMO and VPs) was apparent from the fact that the new bands observed in the right portion of FIG. 3 correspond in molecular weight to the processed proteins. Western blot analysis using anti-His and anti-VP1 were performed and confirmed that result (FIG. 4). FIGS. 3 and 4 also appear to show some cleavage of the His-SUMO from the VPs portion prior to the digestion using SUMO protease.

Sequence listing

Nucleotide sequence encoding HRV14 SUMO-VP0
(LVL1053-43)
SEQ ID NO: 1
atgggtcatcaccatcatcatcacgggtccctgcaggactcagaagtcaa
tcaagaagctaagccagaggtcaagccagaagtcaagcctgagactcaca
tcaatttaaaggtgtccgatggatcttcagagatcttcttcaagatcaaa
aagaccactcctttaagaaggctgatggaagcgttcgctaaaagacaggg
taaggaaatggactccttaacgttcttgtacgacggtattgaaattcaag
ctgatcagacccctgaagatttggacatggaggataacgatattattgag
gctcacagagaacagattggaggtggcgcccaggtgagcacccagaagag
cggcagccacgagaaccagaacatcctgaccaacggcagcaaccagacct
tcaccgtgatcaactactacaaggacgccgccagcaccagcagcgccggc
cagagcctgagcatggaccccagcaagttcaccgagcccgtgaaggacct
gatgctgaagggcgcccccgccctgaacagccccaacgtggaggcctgcg
gctacagcgaccgggtgcagcagatcaccctgggcaacagcaccatcacc
acccaggaggccgccaacgccgtggtgtgctacgccgagtggcccgagta
cctgcccgacgtggacgccagcgacgtgaacaagaccagcaagcccgaca
ccagcgtgtgccggttctacaccctggacagcaagacctggaccaccggc
agcaagggctggtgctggaagctgcccgacgccctgaaggacatgggcgt
gttcggccagaacatgttcttccacagcctgggccggagcggctacaccg
tgcacgtgcagtgcaacgccaccaagttccacagcggctgcctgctggtg
gtggtgatccccgagcaccagctggccagccacgagggcggcaacgtgag
cgtgaagtacaccttcacccaccccggcgagcggggcatcgacctgagca
gcgccaacgaggtgggcggccccgtgaaggacgtgatctacaacatgaac
ggcaccctgctgggcaacctgctgatcttcccccaccagttcatcaacct
gcggaccaacaacaccgccaccatcgtgatcccctacatcaacagcgtgc
ccatcgacagcatgacccggcacaacaacgtgagcctgatggtgatcccc
atcgcccccctgaccgtgcccaccggcgccacccccagcctgcccatcac
cgtgaccatcgcccccatgtgcaccgagttcagcggcatccggagcaaga
gcatcgtgccccagtgataa
Amino acid sequence of HRV14 SUMO-VP0
(LVL1053-43)
SEQ ID NO: 2
MGHHHHHHGSLQDSEVNQEAKPEVKPEVKPETHINLKVSDGSSEIFFKIK
KTTPLRRLMEAFAKRQGKEMDSLTFLYDGIEIQADQTPEDLDMEDNDIIE
AHREQIGGGAQVSTQKSGSHENQNILTNGSNQTFTVINYYKDAASTSSAG
QSLSMDPSKFTEPVKDLMLKGAPALNSPNVEACGYSDRVQQITLGNSTIT
TQEAANAVVCYAEWPEYLPDVDASDVNKTSKPDTSVCRFYTLDSKTWTTG
SKGWCWKLPDALKDMGVFGQNMFFHSLGRSGYTVHVQCNATKFHSGCLLV
VVIPEHQLASHEGGNVSVKYTFTHPGERGIDLSSANEVGGPVKDVIYNMN
GTLLGNLLIFPHQFINLRTNNTATIVIPYINSVPIDSMTRHNNVSLMVIP
IAPLTVPTGATPSLPITVTIAPMCTEFSGIRSKSIVPQ
Nucleotide sequence encoding HRV14 SUMO-VP3
(LVL1054-21)
SEQ ID NO: 3
atgggtcatcaccatcatcatcacgggtccctgcaggactcagaagtcaa
tcaagaagctaagccagaggtcaagccagaagtcaagcctgagactcaca
tcaatttaaaggtgtccgatggatcttcagagatcttcttcaagatcaaa
aagaccactcctttaagaaggctgatggaagcgttcgctaaaagacaggg
taaggaaatggactccttaacgttcttgtacgacggtattgaaattcaag
ctgatcagacccctgaagatttggacatggaggataacgatattattgag
gctcacagagaacagattggaggtggcctgcccaccaccaccctgcccgg
cagcggccagttcctgaccaccgacgaccggcagagccccagcgccctgc
ccaactacgagcccaccccccggatacacatccccggcaaggtgcacaac
ctgctggagatcatccaggtggacaccctgatccccatgaacaacaccca
caccaaggacgaggtgaacagctacctgatccccctgaacgccaaccggc
agaacgagcaggtgttcggcaccaacctgttcatcggcgacggcgtgttc
aagaccaccctgctgggcgagatcgtgcagtactacacccactggagcgg
cagcctgcggttcagcctgatgtacaccggccccgccctgagcagcgcca
agctgatcctggcctacaccccccccggcgcccggggcccccaggaccgg
cgggaggccatgctgggcacccacgtggtgtgggacatcggcctgcagag
caccatcgtgatgaccatcccctggaccagcggcgtgcagttccggtaca
ccgaccccgacacctacaccagcgccggcttcctgagctgctggtatcag
accagcctgatcctgccccccgagaccaccggccaggtgtacctgctgag
cttcatcagcgcctgccccgacttcaagctgcggctgatgaaggacaccc
agaccatcagccagaccgtggccctgaccgagtgataa
Amino acid sequence of HRV14 SUMO-VP3 (LVL1054-21)
SEQ ID NO: 4
MGHHHHHHGSLQDSEVNQEAKPEVKPEVKPETHINLKVSDGSSEIFFKIK
KTTPLRRLMEAFAKRQGKEMDSLTFLYDGIEIQADQTPEDLDMEDNDIIE
AHREQIGGGLPTTTLPGSGQFLTTDDRQSPSALPNYEPTPRIHIPGKVHN
LLEIIQVDTLIPMNNTHTKDEVNSYLIPLNANRQNEQVFGTNLFIGDGVF
KTTLLGEIVQYYTHWSGSLRFSLMYTGPALSSAKLILAYTPPGARGPQDR
REAMLGTHVVWDIGLQSTIVMTIPWTSGVQFRYTDPDTYTSAGFLSCWYQ
TSLILPPETTGQVYLLSFISACPDFKLRLMKDTQTISQTVALTE
Nucleotide sequence encoding HRV14 SUMO-VP1
(LVL1055-12)
Seq ID NO: 5
atgggtcatcaccatcatcatcacgggtccctgcaggactcagaagtcaa
tcaagaagctaagccagaggtcaagccagaagtcaagcctgagactcaca
tcaatttaaaggtgtccgatggatcttcagagatcttcttcaagatcaaa
aagaccactcctttaagaaggctgatggaagcgttcgctaaaagacaggg
taaggaaatggactccttaacgttcttgtacgacggtattgaaattcaag
ctgatcagacccctgaagatttggacatggaggataacgatattattgag
gctcacagagaacagattggaggtggcctgggcgacgagctggaggaggt
gatcgtggagaagaccaagcagaccgtggccagcatcagcagcggcccca
agcacacccagaaggtgcccatcctgaccgccaacgagaccggcgccacc
atgcccgtgctgcccagcgacagcatcgagacccggaccacctacatgca
cttcaacggcagcgagaccgacgtggagtgcttcctgggccgggccgcct
gcgtgcacgtgaccgagatccagaacaaggacgccaccggcatcgacaac
caccgggaggccaagctgttcaacgactggaagatcaacctgagcagcct
ggtgcagctgcggaagaagctggagctgttcacctacgtgcggttcgaca
gcgagtacaccatcctggccaccgccagccagcccgacagcgccaactac
agcagcaacctggtggtgcaggccatgtacgtgccccccggcgcccccaa
ccccaaggagtgggacgactacacctggcagagcgccagcaaccccagcg
tgttcttcaaggtgggcgacaccagccggttcagcgtgccctacgtgggc
ctggccagcgcctacaactgcttctacgacggctacagccacgacgacgc
cgagacccagtacggcatcaccgtgctgaaccacatgggcagcatggcct
tccggatcgtgaacgagcacgacgagcacaagaccctggtgaagatccgg
gtgtaccaccgggccaagcacgtggaggcctggattccccgggccccccg
ggccctgccctacaccagcatcggccggaccaactaccccaagaacaccg
agcccgtgatcaagaagcggaagggcgacatcaagagctactgataa
Amino acid sequence of HRV14 SUMO-VP1 (LVL1055-12)
SEQ ID NO: 6
MGHHHHHHGSLQDSEVNQEAKPEVKPEVKPETHINLKVSDGSSEIFFKIK
KTTPLRRLMEAFAKRQGKEMDSLTFLYDGIEIQADQTPEDLDMEDNDIIE
AHREQIGGGLGDELEEVIVEKTKQTVASISSGPKHTQKVPILTANETGAT
MPVLPSDSIETRTTYMHFNGSETDVECFLGRAACVHVTEIQNKDATGIDN
HREAKLFNDWKINLSSLVQLRKKLELFTYVRFDSEYTILATASQPDSANY
SSNLVVQAMYVPPGAPNPKEWDDYTWQSASNPSVFFKVGDTSRFSVPYVG
LASAYNCFYDGYSHDDAETQYGITVLNHMGSMAFRIVNEHDEHKTLVKIR
VYHRAKHVEAWIPRAPRALPYTSIGRTNYPKNTEPVIKKRKGDIKSY
>sp|P03303|2-331-VP0-HRV14
SEQ ID NO: 14
GAQVSTQKSGSHENQNILTNGSNQTFTVINYYKDAASTSSAGQSLSMDPS
KFTEPVKDLMLKGAPALNSPNVEACGYSDRVQQITLGNSTITTQEAANAV
VCYAEWPEYLPDVDASDVNKTSKPDTSVCRFYTLDSKTWTTGSKGWCWKL
PDALKDMGVFGQNMFFHSLGRSGYTVHVQCNATKFHSGCLLVVVIPEHQL
ASHEGGNVSVKYTFTHPGERGIDLSSANEVGGPVKDVIYNMNGTLLGNLL
IFPHQFINLRTNNTATIVIPYINSVPIDSMTRHNNVSLMVIPIAPLTVPT
GATPSLPITVTIAPMCTEFSGIRSKSIVPQ
>sp|P03303|568-856-VP1-HRV14
SEQ ID NO: 15
GLGDELEEVIVEKTKQTVASISSGPKHTQKVPILTANETGATMPVLPSDS
IETRTTYMHFNGSETDVECFLGRAACVHVTEIQNKDATGIDNHREAKLFN
DWKINLSSLVQLRKKLELFTYVRFDSEYTILATASQPDSANYSSNLVVQA
MYVPPGAPNPKEWDDYTWQSASNPSVFFKVGDTSRFSVPYVGLASAYNCF
YDGYSHDDAETQYGITVLNHMGSMAFRIVNEHDEHKTLVKIRVYHRAKHV
EAWIPRAPRALPYTSIGRTNYPKNTEPVIKKRKGDIKSY
>sp|P03303|70-331-VP2-HRV14
SEQ ID NO: 16
SPNVEACGYSDRVQQITLGNSTITTQEAANAVVCYAEWPEYLPDVDASDV
NKTSKPDTSVCRFYTLDSKTWTTGSKGWCWKLPDALKDMGVFGQNMFFHS
LGRSGYTVHVQCNATKFHSGCLLVVVIPEHQLASHEGGNVSVKYTFTHPG
ERGIDLSSANEVGGPVKDVIYNMNGTLLGNLLIFPHQFINLRTNNTATIV
IPYINSVPIDSMTRHNNVSLMVIPIAPLTVPTGATPSLPITVTIAPMCTE
FSGIRSKSIVPQ
>sp|P03303|332-563-VP3-HRV14
SEQ ID NO: 17
GLPTTTLPGSGQFLTTDDRQSPSALPNYEPTPRIHIPGKVHNLLEIIQVD
TLIPMNNTHTKDEVNSYLIPLNANRQNEQVFGTNLFIGDGVFKTTLLGEI
VQYYTHWSGSLRFSLMYTGPALSSAKLILAYTPPGARGPQDRREAMLGTH
VVWDIGLQSTIVMTIPWTSGVQFRYTDPDTYTSAGFLSCWYQTSLILPPE
TTGQVYLLSFISACPDFKLRLMKDTQTISQTVALTE
>sp|P03303|2-69-VP4-HRV14
SEQ ID NO: 18
GAQVSTQKSGSHENQNILTNGSNQTFTVINYYKDAASTSSAGQSLSMDPS
KFTEPVKDLMLKGAPALN

1.-2. (canceled)

3. A Virus-Like Particle (VLP), comprising human rhinovirus capsid proteins VP0, VP1 and VP 3.

4. The VLP of claim 3, further comprising capsid protein VP 4.

5. The VLP of claim 3, wherein all capsid proteins originate from the same seroptype of human rhinovirus.

6. The VLP of claim 3, wherein the capsid proteins originate from two or more different serotypes of human rhinovirus.

7. The VLP of claim 3, wherein one or more of the capsid proteins comprises at least one modification which increases the ability of the VLP to induce a cross-reactive immune response against multiple human rhinoviral serotypes, as compared to a VLP wherein said modification is not present, where said modification consists of insertion of at least one peptide from a rhinoviral capsid protein, said peptide capable of inducing a cross-reactive immune response against two or more human rhinoviral serotypes.

8.-10. (canceled)

11. The VLP of claim 7, wherein said peptide comprises a sequence selected from (a) amino acids 32-45 from VP1 SEQ ID NO:15, (b) amino acids 32-45 of VP1 SEQ ID NO:15 having 1-4 amino acid deletions at either end, (c) amino acids 32-45 of VP1 SEQ ID NO:15 having 1-2 amino acid substitutions or additions or deletions, (d) SEQ ID NO: 7, (e) SEQ ID NO:8, (f) SEQ ID NO:9, and (q) SEQ ID NO:10.

12. (canceled)

13. The VLP of claim 7, wherein said peptide comprises a sequence selected from (a) amino acids 1-16 from VP4 SEQ ID NO:18, (b) amino acids 1-16 of VP4 SEQ ID NO: 18 having 1-4 amino acid deletions at either end, (c) amino acids 1-16 of VP4 SEQ ID NO:18 having 1-2 amino acid substitutions or additions or deletions, (d) SEQ ID NO:11, (e) SEQ ID NO:12, and (f) SEQ ID NO:13.

14. (canceled)

15. The VLP of claim 7, wherein said peptide is selected from:
(a) amino acids 147-162 of VP1 SEQ ID NO:15,
(b) amino acids 147-162 of VP1 SEQ ID NO:15, having 1-4 amino acid additions or deletions at either end,
(c) amino acids 147-162 of VP1 SEQ ID NO:15, having 1-2 amino acid substitutions or additions or deletions within the peptide sequence,
(d) amino acids 1-30 of VP4 SEQ ID NO:18,
(e) amino acids 1-30 of VP4 SEQ ID NO:18, having 1-4 amino acid additions or deletions at either end,
(f) amino acids 1-30 of VP4 SEQ ID NO:18, having 1-2 amino acid substitutions or additions or deletions within the peptide sequence,
(g) amino acids 1-24 of VP4 SEQ ID NO:18,
(h) amino acids 1-24 of VP4 SEQ ID NO:18, having 1-4 amino acid additions or deletions at either end,
(i) amino acids 1-24 of VP4 SEQ ID NO:18, having 1-2 amino acid substitutions or additions or deletions within the peptide sequence,
(j) amino acids 1-8 of VP1 SEQ ID NO:15,
(k) amino acids 1-8 of VP1 SEQ ID NO:15, having 1-4 amino acid additions or deletions at either end,
(l) amino acids 1-8 of VP1 SEQ ID NO:15, having 1-2 amino acid substitutions or additions or deletions within the peptide sequence,
(m) amino acids 277-283 of VP1 SEQ ID NO:15,
(n) amino acids 277-283 of VP1 SEQ ID NO:15, having 1-4 amino acid additions or deletions at either end,
(o) amino acids 277-283 of VP1 SEQ ID NO:15, having 1-2 amino acid substitutions or additions or deletions within the peptide sequence,
(p) amino acids 275-285 of VP1 SEQ ID NO:15,
(q) amino acids 275-285 of VP1 SEQ ID NO:15, having 1-4 amino acid additions or deletions at either end, and
(r) amino acids 275-285 of VP1 SEQ ID NO:15, having 1-2 amino acid substitutions or additions or deletions within the peptide sequence.

16.-25. (canceled)

26. The VLP of claim 3, wherein the VLP comprises at least one modification which results in attenuation of an immunodominant epitope.

27. The VLP of claim 26, wherein VP0 comprises a modification selected from: (a) attenuation of the NIm-II site epitope, (b) modification of one or more residues in the regions corresponding to amino acids 131-175, 228-238 or 255-262 in VP2 SEQ ID NO:16, and (c) modification of a residue corresponding to residue 158, 159, 161 and/or 162 of VP2 SEQ ID NO:16.

28. The VLP of claim 26, wherein VP1 comprises a modification selected from: (a) attenuation of the NIm-IA site epitope (b) attenuation of the NIm-IB site epitope (c) modification of one or more residues in the regions corresponding to amino acids 73-102, 129-149, 204-212 and 264-289 in VP1 SEQ ID NO:15, and (d) modification of a residue corresponding to residue 83, 85, 91, 95, 138 or 139 in VP1 SEQ ID NO:15.

29. The VLP of claim 26, wherein VP2 comprises a modification selected from: (a) attenuation of the NIm-II site epitope, (b) modification of one or more residues in the regions corresponding to amino acids 131-175, 228-238 or 255-262 in VP2 SEQ ID NO:16, and (c) modification of a residue corresponding to residue 158, 159, 161 or 162 in VP2 SEQ ID NO:16.

30. The VLP of claim 26, wherein VP3 comprises a modification selected from: (a) attenuation of the NIm-Ill site epitope, (b) modification of one or more residues in the regions corresponding to amino acids 54-95, 193-212 or 226-236 in VP3 of SEQ ID NO: 17, and (c) modification of a residue corresponding to residue 72, 75 or 78 in VP3 SEQ ID NO: 17.

31. The VLP of claim 4, wherein the VLP comprises at least one modification in capsid protein VP4 which results in attenuation of an immunodominant epitope.

32. The VLP of claim 3, wherein the VP0 protein has a sequence selected from the group consisting of (a) SEQ ID NO:14; (b) a sequence having more than 90% sequence identity to SEQ ID NO:14, (c) SEQ ID NO:14 having from 1-25 amino acid additions or deletions at either end, and (d) SEQ ID NO:14 having 1-50 amino acid substitutions or deletions within the sequence.

33. (canceled)

34. The VLP of claim 3, wherein the VP1 protein has a sequence selected from the group consisting of (a) SEQ ID NO:15, (b) a sequence having more than 90% sequence identity to SEQ ID NO:15, (c) SEQ ID NO:15 having from 1-25 amino acid additions or deletions at either end, and (d) SEQ ID NO:15 having 1-50 amino acid substitutions or deletions within the sequence.

35. (canceled)

36. The VLP of claim 3, wherein the VP2 protein has a sequence selected from the group consisting of (a) SEQ ID NO:16, (b) a sequence having more than 90% sequence identity to SEQ ID NO:16, (c) SEQ ID NO:16 having from 1-25 amino acid additions or deletions at either end, and (d) SEQ ID NO:16 having 1-50 amino acid substitutions or deletions within the sequence.

37. (canceled)

38. The VLP of claim 3, wherein the VP3 protein has a sequence selected from the group consisting of (a) SEQ ID NO: 17, (b) a sequence having more than 90% sequence identity to SEQ ID NO:17, (c) SEQ ID NO:17 having from 1-25 amino acid additions or deletions at either end, and (d) SEQ ID NO:17 having 1-50 amino acid substitutions or deletions within the sequence.

39. (canceled)

40. The VLP of claim 3, wherein the VP4 protein has a sequence selected from the group consisting of (a) SEQ ID NO:18, (b) a sequence having more than 90% sequence identity to HRV14 VP4 SEQ ID NO:18, (c) SEQ ID NO:18 having from 1-25 amino acid additions or deletions at either end, and (d) SEQ ID NO:18 having 1-50 amino acid substitutions or deletions within the sequence.

41. (canceled)

42. An immunogenic composition comprising the VLP of claim 3 and a pharmaceutically acceptable diluent, excipient or carrier.

43.-48. (canceled)

49. A method for inducing an immune response in a human against human rhinovirus comprising administering to said human an immunogenic composition according to claim 42.

50.-53. (canceled)

54. A method for producing rhinoviral VLPs comprising the steps of:
(a) producing constructs encoding capsid proteins required for the formation of a VLP, wherein each of the capsid proteins is encoded as a fusion protein with a chaperone protein, and
(b) expressing said constructs in a host cell, and
(c) lysing said host cells.

55. (canceled)

56. The method according to claim 54, wherein said chaperone protein is a Small Ubiquitin-related Modifier (SUMO) protein.

57.-67. (canceled)
